(19) United States
(12) Patent Application Publication
(10) Pub. No.: US 2006/0129164 A1
(43) Pub. Date: Jun. 15, 2006

(54) Medical instrument for manipulating objects that have a hollow channel

(76) Inventor: Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/266,693

(22) Filed: Nov. 3, 2005

(30) Foreign Application Priority Data
Nov. 3, 2004 (DE) 10 2004 053 466.7

(51) Int. Cl. A61F 2/36

(52) U.S. Cl. 606/104

(57) ABSTRACT

A medical instrument is used for manipulating objects that have a hollow channel. The medical instrument has a handle at the proximal end and a distal tip that can be inserted into the hollow channel of the object to be manipulated. An expander mechanism is provided for the radial expansion of the distal tip to such an extent that the tip and the object can be pressed tightly together.

### PRIOR APPLICATIONS

This application claims priority of German Patent Application No. 10 2004 053 466.7 filed on Nov. 3, 2004.

### FIELD OF THE INVENTION

The invention relates to a medical instrument for manipulating objects that have a hollow channel.

### BACKGROUND OF THE INVENTION

Objects that have a hollow channel include implants that are used in the human body, particularly in bones. Conventional implants, in the form of what are known as interference screws, are used to fix a tendon replacement in place, particularly a cruciate ligament replacement in the knee. To do this, a suitable hole is drilled in the femur or tibia and the tendon replacement is inserted into it. To fix the tendon in place, a so-called interference screw is screwed into the free space between the tendon and the wall of the hole, pressing the tendon laterally against the wall of the hole and thus fixing it in place.

Other objects include suture anchors, inserted into holes drilled in bone to fix holding or fixation sutures in place.

What all these objects have in common, is that they have a hollow channel. This hollow channel may take the form of a channel through the entire axial length of the object, e.g. to allow fixation sutures to be threaded through the body, or it may simply take the form of a socket to permit the use of a tool, e.g. a screwdriver, to rotate the object, if it is in the form of an interference screw.

The hollow channel can have any cross-sectional shape, for example a round cross section, a hexagonal or a star-shaped cross section.

It is general knowledge that such interference screws may be either made of metals, particularly titanium, or of biodegradable materials, see OP-Journal, Special Edition, No. 3/Volume 14/December 1998, A. Weiler et al. “Biodegradierbare Interferenzschrauben in der Kreuzbandchirurgie [Biodegradable Interference Screws in Cruciate Ligament Surgery]”.

A surgical technique, in particular the abovementioned fixation of a tendon transplant, is described for example in EP 1 093 773 A1 by the applicant.

In practical use, it has been found that fixation of such objects involves relatively large forces, which can lead to breakage or deformation of the corresponding slits or hexagonal socket in which a rotary tool is fitted.

Another problem occurs particularly in cruciate ligament surgery in so-called revision surgery, i.e. when the implant needs to be removed again, for example because the tendon has torn again.

In this case, the recovery of the implants used by the previous surgeon is a particular challenge. This is because the implants are overgrown by the surrounding tissue and bone and the screw heads were already deformed during insertion.

Therefore, the object of the present invention is to make available a medical instrument for manipulating objects of this type, which permits manipulation of objects of this type as non-destructively as possible, in particular permits the recovery of such objects during revision surgery.

### SUMMARY OF THE INVENTION

According to the invention, this problem is solved by means of the medical instrument having a handle at the proximal end and a distal tip that can be inserted into a hollow channel of the object to be manipulated together with an expander mechanism for the radial expansion of the distal tip to such an extent that the tip and the object can be pressed tightly together.

The distal tip can now be inserted into the hollow channel of the object to be manipulated. For this purpose the tip is of an appropriate length and an appropriate diameter.

By using the expander mechanism, the distal tip can be expanded radially after insertion into the hollow channel, causing it to press over a wide area and under a pressure against the inner wall of the hollow channel, so that it sits in the object to be manipulated, exerting pressure on the firmly surface.

This results in a relatively large area of firm frictional contact between the object and the medical instrument, permitting the object to be manipulated.

The manipulation may involve either screwing in or insertion of the object or the recovery of the object.

A particular advantage of the expander mechanism is that no matching shape is generated, as is normal for tools, but that a contact for manipulation which is firm when pressure is exerted on the surface is created by means of the expansion.

This makes possible, e.g. in the recovery of interference screws that have already been relatively firmly overgrown with the bone material, the creation of a firm connection between the instrument and the object, so that even correspondingly high forces can be applied to allow a screw that has been overgrown like this to be unscrewed.

Because of the relatively large area of contact in the hollow channel, it is irrelevant whether, for example, a hexagonal channel was already deformed during insertion of the screw, the expansion ensures sufficiently firm contact.

The material from which the device is made can be chosen according to the material from which the object to be recovered is made, whether metal or plastic.

If, for example, the hollow channel shows relatively severe geometric deformation, the material of the distal tip can be relatively soft, so that the expander mechanism makes it deform to fit the deformation, so that a particularly large area of contact is possible. The material can also be metal, in order, in the case of plastic interference screws for example, to smooth out any bumps or ingrows in the region of the hollow channel and thus obtain a large area of tight fit.

Ultimately, a medical instrument of this type can be used with any type of cannulated implant, the preferred use being in the recovery of an implant during revision surgery. The device can, however, also be used for primary implantation. It is no longer necessary to drill out the implant to recover it, which, e.g. in the case of metal implants leads to metal particles that must be carefully removed. From now on, so-called giant defects caused by drilling out implants can also be avoided.

In another embodiment of the invention, the distal tip is formed as a hollow body that can be expanded axially by the expander mechanism.

This measure has the advantage that the parts required for the expansion can be accommodated inside the hollow body in order to expand the latter axially. The entire outer surface of the hollow body can thus be used to form the surface contact.

In another embodiment of the invention, a wall of the hollow body has at least one slit.

This measure has the advantage that the expansion can be carried out in a deliberate and predetermined manner, without permanent deformation of the material being necessary, this being conductive to long-term use of this instrument.

In another embodiment of the invention, there is at least one axial slit.

This measure has the advantage that the hollow body can now be expanded particularly well. Depending on the size, one to four or five slits can be spaced evenly around the circumference, so that correspondingly clearly defined segments can then be expanded axially.

In another embodiment of the invention, the expander mechanism has an expanding mandrel.

This measure has the advantage that the distal tip can initially be inserted into the hollow channel undilated, and then be expanded by the expanding mandrel.

In another embodiment of the invention, the expanding mandrel takes the form of a cone.

This measure has the advantage that this cone geometry can generate a uniform axially expanding pressure over the surface of the cone.

In another embodiment of the invention, the inside of the hollow body has a corresponding conical shape.

This measure has the advantage that the distal tip expands slightly as soon as the conical surfaces come into contact, and this leads to the “tight fit” in the hollow channel over a large surface area.

In another embodiment of the invention, the expanding mandrel has an extension that projects beyond the distal end of the tip.

This measure has the advantage that this extension represents a sort of guide wire or aid for the insertion of the instrument into the hollow channel of the object to be manipulated.

In another embodiment of the invention, a handle is fitted proximally with a knob to operate the expander mechanism.

This measure makes it easier to manipulate the medical instrument. It can, for example, be held in the hand like a screwdriver, and initially positioned, and inserted into the hollow channel of the object to be manipulated, and then the expander mechanism is operated using the knob on the grip.

Another embodiment of the invention has the advantage that the knob has a lever that can move the expanding mandrel axially in the tip.

This measure also makes it easier to use. After positioning and insertion into the hollow channel, the lever is rotated, displacing the expanding mandrel distally and producing the expansion. The operator can also always tell the position of the expander mechanism from the pivot position of the lever.

It goes without saying that the features cited above and described below cannot just be used in the combinations cited, but also in other combinations or on their own, without departing from the scope of the present invention.

### BRIEF DESCRIPTION OF THE DRAWINGS

The invention will be described in more detail below and discussed on the basis of a few selected exemplary embodiments together with the accompanying drawings. They show:

FIG. 1 is a side view of a medical instrument in accordance with the invention in the region of the distal tip,

FIG. 2 is a longitudinal section of the view in FIG. 1,

FIG. 3 is a cross section along the line III-III in FIG. 2,

FIG. 4 is an illustration similar to the sectional illustration in FIG. 2, somewhat enlarged, in which a mandrel of the expander mechanism has already been introduced for expansion,

FIG. 5 is another embodiment of an expanding mandrel of that type with a distal extension,

FIG. 6 is a highly schematically, the medical instrument just before insertion into a hollow channel of a fixation screw fitted in a bone, and

FIG. 7 is a view similar to FIG. 6 after insertion of the distal tip into the hollow channel of the screw and operation of the expander mechanism to produce the tight fit connection between the instrument and the object to be recovered.

### DETAILED DESCRIPTION OF THE INVENTION

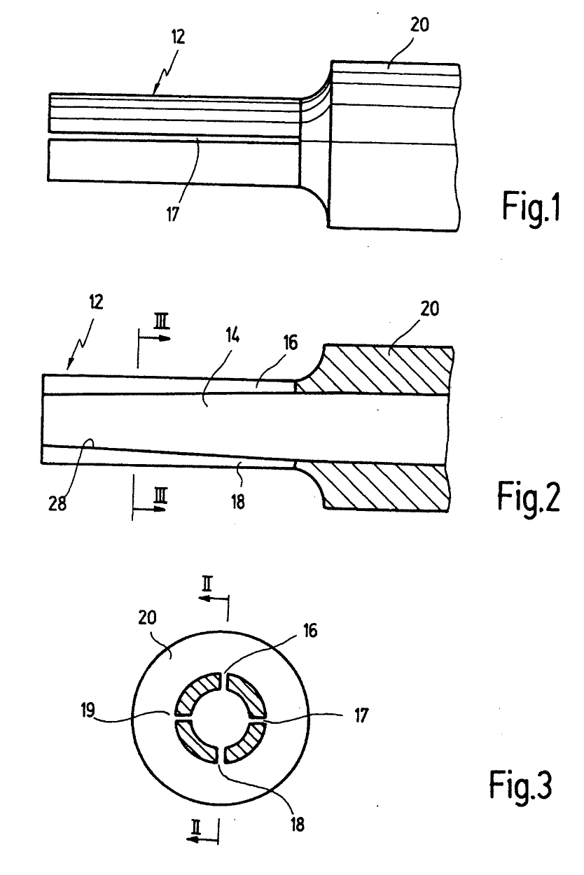

A medical instrument as described in FIGS. 1 to 7 as a whole is designated by reference number 10.

As can be seen from FIGS. 1 to 3 in particular, the medical instrument 10 has a distal tip 12, formed as a hollow body 14.

The hollow body 14 has four evenly spaced slits around the circumference 16, 17, 18, 19 which run axially up to the distal end and go all the way through.

The distal tip 12 is connected to a hollow cylindrical body 20 with a larger diameter.

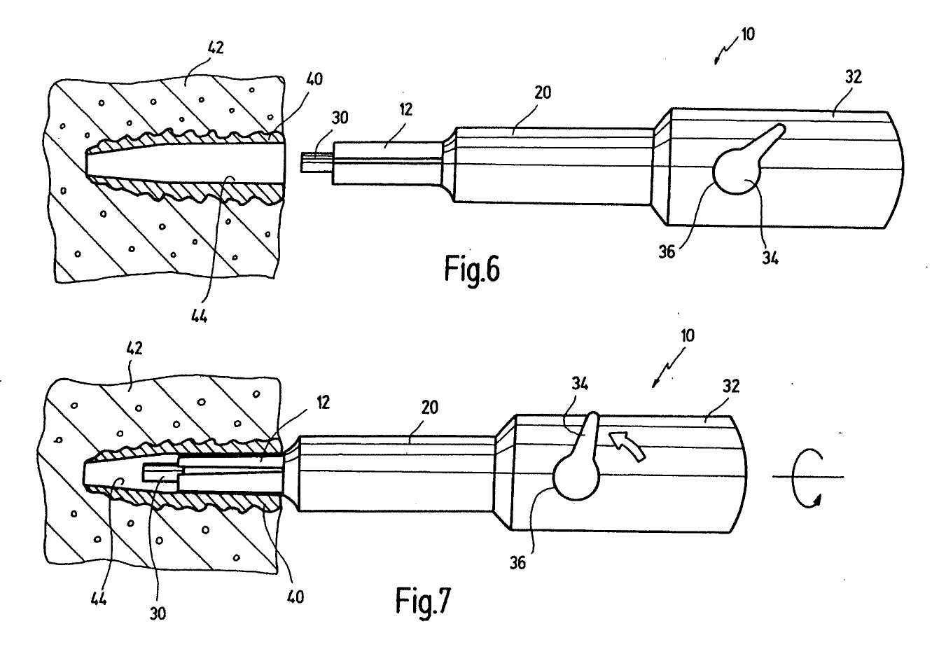

As can be seen in particular from FIGS. 6 and 7 the proximal end of the body 20 joins up to an approximately cylindrical handle 32 of even greater diameter.

Inside the medical instrument 10, which is hollow throughout its length there is an expanding mandrel 22. The expanding mandrel 22 has a cylindrically rod-shaped body 24, the distal end region of which takes the form of a cone 26.

The inside 28 of the hollow body 14 has a conical shape complementary to the cone 26.

The rod-shaped body 24 extends into the handle 32, where it is operatively connected to a lever 34 which can move the expanding mandrel 22 axially forwards and backwards.

FIG. 5 shows another embodiment of an expanding mandrel 22′, which only differs from the expanding mandrel 22 in that its distal end is extended by a guide rod 30 or guide wire. The length of this guide section is such that it extends beyond the distal end of the distal tip 12, as can be seen from FIG. 6.

The lever and expanding mandrel components make up an expander mechanism 36, which serves to expand the hollow body 14 radially.

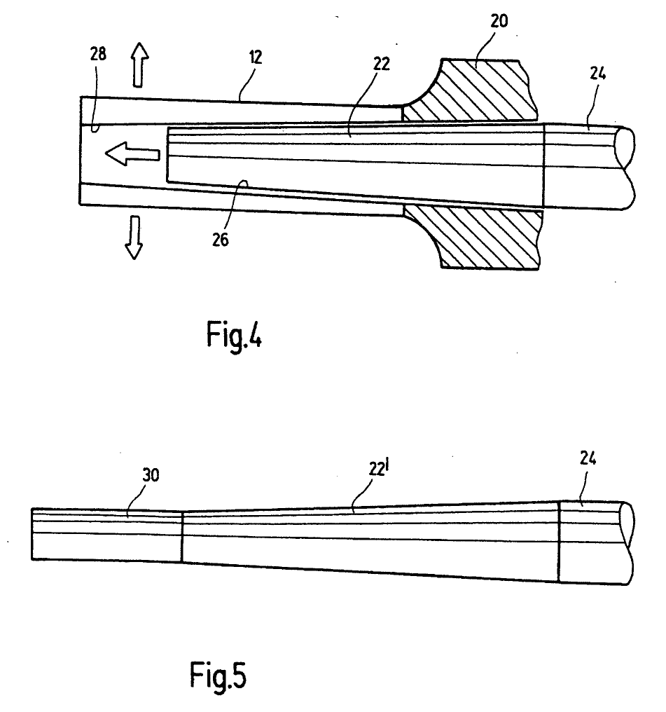

As can be seen from FIG. 4 in particular, the cone 26 and the correspondingly conical inner side 28 of the hollow body 14 come into contact with one another, after which advancing the expanding mandrel 22 further in the distal direction makes the hollow body 14 expand radially or widen, as illustrated by the arrows in FIG. 4. This expansion or widening is made easier in particular by the hollow body 14 being divided into four segments by the slits 16 to 19, as can be seen particularly well in the sectional illustration in FIG. 3.

In conjunction with FIGS. 6 and 7, a practical application will now be illustrated, in this case the medical instrument 10 serving to recover an object 40, which takes the form of an interference screw, which has been screwed into a bone 42. In the middle of the object 40 there is a cylindrical hollow channel 44.

In this case, the geometry of the distal tip 12 is also cylindrical, and the outside diameter of the hollow body 14 in the unexpanded state corresponds roughly to the free inner diameter of the hollow channel 44.

In the case of other relevant sizes or other relevant geometries, the geometry of the distal tip 12 and hollow channel 44 are correspondingly matched to one another.

In the position shown in FIG. 6, the expander mechanism 36 is in its unexpanded position, i.e. the expanding mandrel 22 is just touching the inner conical surface of the hollow body 14.

The guide rod or guide wire 30 projects a little way beyond the hollow body 14 and serves to make it easier to position and thread or insert the distal tip 12 into the hollow channel of the object to be recovered 40.

After the hollow body 14 has been inserted all the way into the hollow channel 44, the lever 34 is rotated, which pushes the expanding mandrel 22 in a distal direction and radially expands and widens the segments of the hollow body 14, producing firm surface pressure contact between the expanded segments of the hollow body 14 and the inside wall of the hollow channel 44 of the object to be recovered 40.

This assembly can now be rotated, for example anticlockwise, to unscrew the fixation screw from the bone and recover it.

It is obvious that such an assembly can also be used to insert a screw of this type. In that case, the screw, or rather its hollow channel, is slipped onto hollow body 14, the expander mechanism is operated, and this assembly is then positioned in an appropriate drilled hole, to insert the screw.

The expander mechanism is then correspondingly operated again, so that the medical instrument 10 can then be removed.

What is claimed is:

1. A medical instrument for manipulating an object having a hollow channel, comprising
a handle at a proximal end,
a distal tip at a distal end, said distal tip can be inserted into said hollow channel of said object to be manipulated, and
an expander mechanism for radially expanding said distal tip to such an extent that said distal tip and said object are pressed tightly together.

2. The medical instrument of claim 1, wherein said distal tip is designed as a hollow body that can be expanded axially by said expander mechanism.

3. The medical instrument of claim 2, wherein a wall of said hollow body has at least one slit.

4. The medical instrument of claim 3, wherein said at least one slit takes the form of an axial slit.

5. The medical instrument of claim 1, wherein said expander mechanism comprises an expanding mandrel.

6. The medical instrument of claim 5, wherein said expanding mandrel has a cone.

7. The medical instrument of claim 6, wherein an inner side of a hollow body of said distal tip has a conical shape corresponding to said cone of said extending mandrel.

8. The medical instrument of claim 1, wherein said expander mechanism has an expanding mandrel having an extension that projects beyond a distal end of said distal tip.

9. The medical instrument of claim 1, wherein said handle has a knob for operating said expander mechanism.

10. The medical instrument of claim 9, wherein said knob has a lever which can move an expanding mandrel in axially direction within said distal tip.
